(19) United States
(12) Patent Application Publication
(10) Pub. No.: US 2009/0162675 A1
(43) Pub. Date: Jun. 25, 2009

(54) PLASTIC PACKAGING ARTICLES FOR OXYGEN SENSITIVE PRODUCTS

(75) Inventor: Philip Bourgeois, Perrysburg, OH (US)

Correspondence Address: RISSMAN HENDRICKS & OLIVERIO, LLP, 100 Cambridge Street, Suite 2101, BOSTON, MA, 02114, US

(73) Assignee: Graham Packaging Co., York, PA (US)

(21) Appl. No.: 11/961,053

(22) Filed: Dec. 20, 2007

(51) Int. Cl. C07C 35/18; C01B 3/00; B32B 15/08; B32B 15/088

(52) U.S. Cl. 428/454; 252/188.28; 428/457; 428/458; 428/461; 568/826

(57) ABSTRACT

Plastic packaging articles having an oxygen-scavenging composition, comprising:
a compound comprising a ring having three ring carbon atoms forming an allyl group with an allylic hydrogen, the ring being bonded via a ring substituent to an ether or carboxylic linkage, the ether or carboxylic linkage being further bonded to a group having a molecular weight of at least 75 g/mol; and
at least one transition metal.

### FIELD OF THE INVENTION

The present invention relates to plastic articles, such as preforms, containers, liners, flexible films, closures, and other materials for the packaging of oxygen-sensitive products.

### BACKGROUND OF THE INVENTION

In the development of plastic bottles for containing oxygen-sensitive food and beverages, e.g., beer, juice, ketchup, generally two types of oxygen barrier materials have been used. A “passive” barrier retards oxygen permeation into the package. Exemplary passive barrier materials include polyvinylidene chloride copolymer (PVDC), ethylene vinyl alcohol copolymer (EVOH), and MXD6. These can be used in the manufacture of packaging-grade plastic resins (e.g., polyethylene terephthalate (PET), polypropylene, and polyethylene).

An “active” barrier, also known as an oxygen “scavenger,” can be incorporated into a single or multi-layer plastic structure to remove the oxygen initially present and/or generated from the inside of the package, as well as to retard the passage of exterior oxygen into the package. Oxygen scavengers have advantages in some regard to passive barriers in that they remove oxygen from inside the package, as well as retard its ingress into the package.

Typical oxygen-scavenging compositions include polymers that can be oxidized, such as m-xylylenediamine-nylon (MXD-6 nylon), with the aid of a transition metal (e.g., cobalt). For example, U.S. Pat. No. 5,021,515 describes a bottle formed from a blend of PET and MXD-6. However, it is well known in the art that these blends of PET and MXD-6, due to solubility issues, may cause aesthetic and/or physical issues.

Accordingly, there remains a need to develop transparent plastic articles for holding oxygen-sensitive food and other products.

### SUMMARY OF THE INVENTION

One embodiment of the invention provides an oxygen scavenging composition for incorporation into a plastic container that does not result in a significant loss of clarity, e.g., having low haze.

In one embodiment, a composition is provided comprising at least one transition metal, and a compound having the following structure:

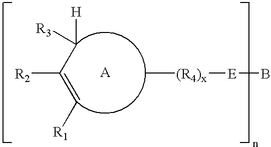

wherein “A” is a hydrocarbon ring; R₁, R₂, and R₃ are independently selected from hydrogen and C₁-C₁₂ alkyl; R₄ is an alkenyl of the formula —CH₂)_{y}—, where y is an integer of at least 1, and x is an integer equal to 1 or 0; E is selected from —O—, —C(═O)—O—, and —N(R)— where R is hydrogen, aryl or a branched or linear C₁-C₆ alkyl; B is a group having a molecular weight of at least 75 g/mol; and n is an integer ranging from 1-3.

More specifically, B may be a hydrocarbon comprising at least 6 carbon atoms. B may be a hydrocarbon selected from alkyl, aryl, and combinations of alkyl and aryl groups.

R₄ may be a C₁-C₁₂ alkenyl. A may be a C₆ ring. The value of n may be: n=1 or n=2 or 3.

In one embodiment, a plastic packaging article is provided having at least one layer comprising a polymeric material blended with an the oxygen-scavenging composition, the composition comprising: at least one transition metal, and a compound having the following structure:

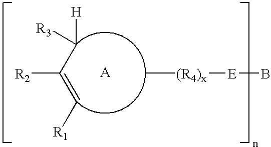

wherein: “A” is a hydrocarbon ring; R₁, R₂, and R₃ are independently selected from hydrogen and C₁-C₁₂ alkyl; R₄ is an alkenyl of the formula —(CH₂)_{y}—, where y is an integer of at least 1, and x is an integer equal to 1 or 0; E is selected from —O—, —C(═O)—O—, and —N(R)— where R is hydrogen, aryl or a branched or linear C₁-C₆ alkyl, B is a group having a molecular weight of at least 75 g/mol; and n is an integer ranging from 1-3.

The polymeric material may comprise a polymer selected from polyesters, polyolefins, polyacrylates, polyvinyl acetates, styrenic polymers, EVOH, polyamides, polyacrylamides, polyacrylonitriles, poly(styrene-co-acrylonitrile), poly(vinyl chloride), poly(vinylidene chloride), polytetrafluoroethylene, polycarbonates, polyethers, polyimides, polybenzimidazoles, polybenzoxazoles, poly(vinyl pyrrolidone)), polysulfones, poly(ether ether ketones), poly(phenylene oxide), poly(phenylene sulfide), polysiloxanes, polysilanes, polyphosphazenes), dextran, cellulose, carboxymethyl cellulose, and sulfonate polymers.

The polyester may be PET. The article may be a monolayer article or a multilayer article. The article may further comprise at least one polymer layer free of the oxygen-scavenging composition positioned adjacent the at least one layer containing the oxygen-scavenging composition.

In one embodiment, the article includes at least one polymer layer free of the oxygen-scavenging composition positioned adjacent either side of the at least one polymer layer containing the oxygen-scavenging composition.

The at least one layer containing the oxygen-scavenging composition may comprise PET.

One or more layers, including the at least one layer free of the oxygen-scavenging composition, may comprise a barrier polymer.

The barrier polymer may be selected from EVOH, polyamides, acrylonitrile copolymers, blends of EVOH and polyamide, nanocomposites of EVOH or polyamide and clay, blends of EVOH and an ionomer, acrylonitrile, cyclic olefin copolymers, polyvinylidene chloride (PVDC), polyethylene napthalate (PEN) polyglycolic acid (PGA), and blends thereof.

The article may be a monolayer article or a multilayer article.

The article may be selected from preforms, containers, closures, closure liners, films, and sheets.

In one embodiment, an oxygen-scavenging composition is provided comprising: a nonpolymeric compound comprising a ring, three of the ring carbon atoms forming an allyl group with an allylic hydrogen, the ring being bonded via a ring substituent to an ether or carboxylic linkage, the ether or carboxylic linkage being further bonded to a group having a molecular weight of at least 75 g/mol; and at least one transition metal.

In one embodiment, a plastic packaging article is provided having at least one layer comprising the oxygen-scavenging composition, wherein the article is selected from preforms, containers, closures, closure liners, films, and sheets.

In one embodiment, compound is provided having the following structure:

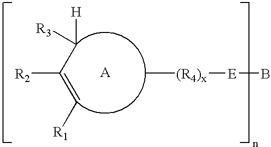

wherein: “A” is a hydrocarbon ring; R₁, R₂, and R₃ are independently selected from hydrogen and C₁-C₁₂ alkyl; R₄ is an alkenyl of the formula —(CH₂)_{y}—, where y is an integer of at least 1, and x is an integer equal to 1 or 0; E is selected from —O—, —C(═O)—O—, and —N(R)— where R is hydrogen, aryl or a branched or linear C₁-C₆ alkyl; B is a group having a molecular weight of at least 75 g/mol, and n is an integer ranging from 1-3.

### DETAILED DESCRIPTION

The present invention relates to oxygen scavenging compositions that can be incorporated into a plastic packaging articles, e.g. performs, containers, closures, closure liners, sheets, and films for protecting oxygen-sensitive products.

In one embodiment, the packaging article (e.g., a preform and its ensuing blow-molded container) can have a monolayer or multilayer arrangement in which at least one layer includes an active oxygen barrier composition, i.e., an oxygen-scavenging composition. The article can further contain one or more layers that function as a passive oxygen barrier.

In one embodiment, the oxygen-scavenging composition includes a nonpolymeric compound comprising a hydrocarbon ring having three ring carbon atoms forming an allyl group with an allylic hydrogen. A “ring atom” such as a “ring carbon” as used herein refers to an atom, e.g., a carbon atom, positioned at a vertex of the ring in which the collection of vertices defines the ring. In contrast, a ring substituent does not define the ring but is attached to the ring itself.

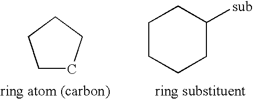

Allyl groups are well known in the art and have the formula (Rₐ)(R_{b})C═C—C(H)(R_{c})(R_{d}), where H is the hydrogen on the allylic carbon atom and is readily reactive and Rₐ-R_{d} are typically alkyl, aryl, and alkenyl groups.

One embodiment provides a compound having the following structure:

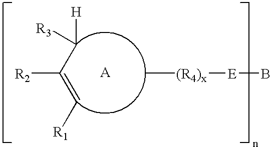

wherein:
“A” is a hydrocarbon ring,
R₁, R₂, and R₃ are independently selected from hydrogen and C₁-C₁₂ alkyl,
R₄ is an alkenyl of the formula —(CH₂)_{y}—, where y is an integer of at least 1, and x is an integer equal to 1 or 0,
E is selected from —O— (ether linkage), —C(═O)—O— (ester linkage) and —N(R)— (amide linkage) where R is hydrogen, aryl or a branched or linear C₁-C₆ alkyl,
B is a group having a molecular weight of at least 75 g/mol, and
n is an integer ranging from 1-3.

In one embodiment, the compound can act as an oxygen scavenger due to the capability of the allylic hydrogen to react with oxygen. Without wishing to be bound by any theory, the mechanism of the oxygen scavenging, in one embodiment, can be represented by the following reaction:

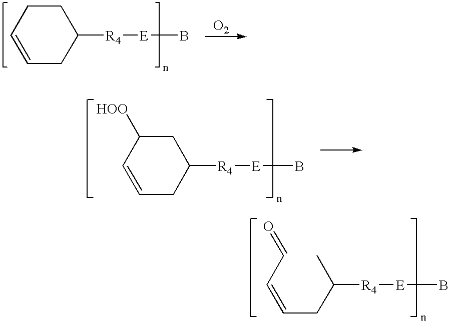

The allylic hydrogen aids in stabilizing the intermediate before reversion to a degradation end product. The reaction with oxygen causes a bond to be broken, producing at least two products. With a cyclic compound, there is a potential of limiting the number of degradation products to one, unless the degradation product can react further with oxygen.

In one embodiment, the oxygen reactivity can be aided with a catalyst, such as a transition metal catalyst. Accordingly, one embodiment of the present invention provides an oxygen-scavenging composition comprising:

a compound having the following structure:

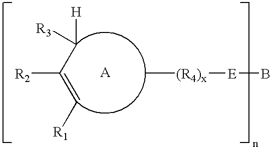

as defined above, and at least one transition metal.

In one embodiment, the transition metal is selected from iron, cobalt, nickel, ruthenium, rhodium, palladium, osmium, iridium, platinum, copper, manganese and zinc. The metal may be added as a salt or as a metal complex. For example, the metal may be added as a complex with an organic ligand such as a carboxylate, an amine, or an alkene. Examples of ligands which may form complexes with the above transition metals include naphthenate, octoate, tallate, resinate, 3,5,5-trimethylhexoate, stearate, palmitate, 2-ethylhexanoate, neodecanoate, acetate, butyrate, oleate, valerate, cyclohexanebutyrate, acetylacetonate, benzaylacetonate, dodecylacetylacetonate, benzoate, oxalate, citrate, tartrate, dialkyldithiocarbamate, disalicylalethylenediamine chelate, and phythalocyanine. Examples of specific metal complexes which may be useful include cobalt (II) 2-ethylhexanoate, cobalt (II) neodecanoate, cobalt (II) acetate, cobalt (II) oleate, and cobalt (II) stearate.

The prior art describes oxygen scavengers where the scavenger is a component of the chemical structure of the polymeric material that forms the oxygen scavenging container, either in the polymeric backbone or as a pendant group. However, the scavenging component is consumed by oxygen, and consumption necessarily causes a change in the chemical structure of the polymeric material, which can change the structural/mechanical properties of the container and may affect the compatibility with other polymers blended or positioned adjacent with the polymeric material. In contrast, the small molecule/nonpolymeric compounds disclosed herein do not constitute a chemical component of the polymeric material of the container and thus, their reaction with oxygen does not change the chemical structure of the polymeric material. Moreover, small molecules are generally easier to tailor than polymers and can be derivatized to have different chemical structures and/or numbers of scavenging units, as disclosed in greater detail below.

In one embodiment, the hydrocarbon ring A is a 5-12 membered ring, such as a 5-8 membered ring. In one embodiment, the hydrocarbon ring A is a 6 membered ring.

“Alkyl” as used herein refers to branched, linear, cyclic (e.g., cycloalkyl), and combinations thereof, e.g., alkylcycloalkyl. In one embodiment, R₁, R₂, and R₃ are independently selected from hydrogen and C₁-C₆ alkyl (branched, linear, cyclic, or combinations thereof.

R₄ is optionally present and is a linear or branched alkylene group capable of linking a hydrocarbon ring with E, where E is an ether oxygen, ester, or an amide group. In one embodiment, R₄ has the formula —(CH₂)_{y}— where y is an integer of at least 1, such as an integer ranging from 1-12, or an integer ranging from 1-6. When R₄ is not present, E is bonded to a ring carbon.

In one embodiment E is an ether oxygen or an ester linkage. The formation of ether and ester linkages are well known in the art, as exemplified in March et al., Advanced Organic Chemistry, Fourth Ed., Wiley, 1992, the disclosure relevant to ether and ester linkages being incorporated herein by reference. For example, ether linkages can be prepared by acid catalyzed (e.g., H₂SO₄) intermolecular dehydration of alcohols, or by the Williamson ether synthesis. In one embodiment, E is an ester group, which can be formed by methods well known in the art, such as the reaction between a carboxylic acid and a hydroxyl group, as described in March et al. above. In yet another embodiment, E is an amide linkage of the formula —N(R)— where R is hydrogen, aryl or a branched or linear C₁-C₆ alkyl.

In one embodiment, B is a large group, e.g., having a molecular weight of at least 75 g/mol. By having a larger molecule as an oxygen scavenger, its mobility can be limited, which can advantageously hamper its ability to migrate into a packaged product due to its size. Additionally, with a large molecular weight, the volatility of the molecule is reduced, which is advantageous when subjected to conditions typically seen during the processing of packaging materials, e.g., elevated temperatures. In one embodiment, B is a hydrocarbon group comprising at least 6 carbon atoms, e.g., alkyl (branched, linear, cyclic, and combinations thereof), aryl, and combinations of alkyl and aryl groups having C₆-C₃₀ carbon atoms. In one embodiment, B is unreactive with molecular oxygen to prevent the formation of excessive byproducts, e.g., B is a saturated hydrocarbon.

In one embodiment, ring A-R₄ has the formula:

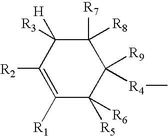

wherein R₁-R₃ and R₅-R₉ are independently selected from hydrogen, C₁-C₁₂ alkyl, and C₆-C₁₂ aryl, and R₄ is defined as disclosed herein. In other embodiments where ring A is other than a 6 membered ring, similar substitution patterns can be envisioned, e.g., three ring carbons forming an allyl group with an available allylic hydrogen, and the remainder of the carbon rings can be independently substituted with hydrogen, C₁-C₁₂ alkyl, or C₆-C₁₂ aryl.

Exemplary compounds where E is an ether linkage (—O—) include, but are not limited to (where Me is methyl (CH₃)):

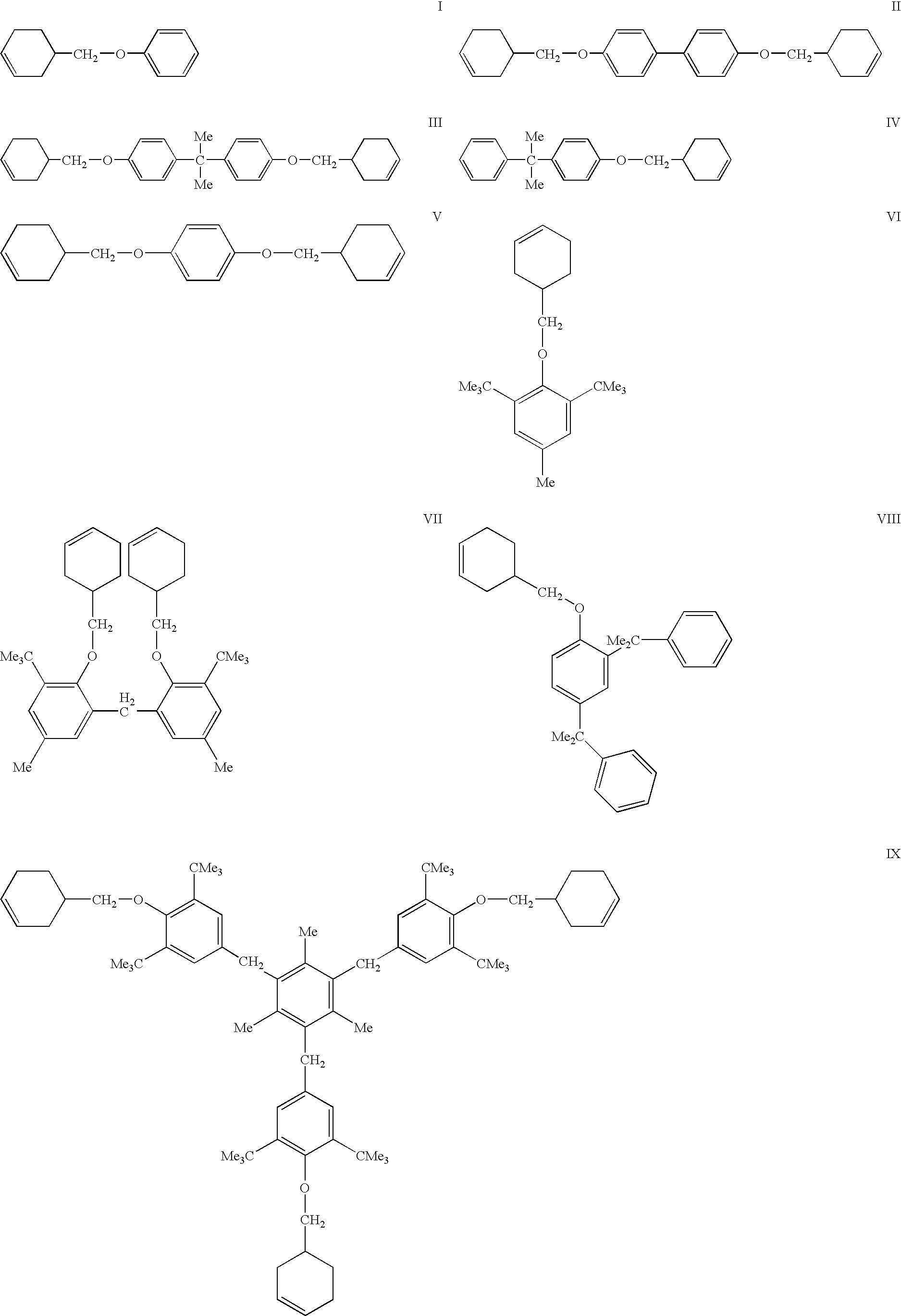

From the examples above, it can be seen that in some cases a single compound can contain one or more A ring groups. A compound can be tailored to have one or more reactive sites that can scavenge one or more oxygen molecules, depending on the number of A ring groups. The number of reactive sites per compound is indicated in Table 1 below.

TABLE 1
Stoichiometry of A ring per compound

| | | | mol | |
|---|---|---|---|---|
| | Formula | MW of R₄ | of A rings/mol | MW of ether |
| No. | of R₄ | (g/mol) | compound | (g/mol) |
| A ring starting | C₇H₁₂O | 112.2 | — | — |
| material (alcohol) | | | | |
| I | C₆H₆O | 94.1 | 1 | 188.3 |
| II | C₁₂H₁₀O₂ | 186.2 | 2 | 374.5 |
| III | C₁₅H₁₆O₂ | 228.3 | 2 | 416.6 |
| IV | C₁₅H₁₆O | 212.3 | 1 | 306.5 |
| V | C₆H₆O₂ | 110.1 | 2 | 298.4 |
| VI | C₁₅H₂₄O | 220.4 | 1 | 314.5 |
| VII | C₂₃H₃₂O₂ | 340.5 | 2 | 528.8 |
| VIII | C₂₄H₂₆O | 330.5 | 1 | 424.6 |
| IX | C₅₄H₇₈O₃ | 775.2 | 3 | 1057.7 |

In one embodiment, the amount of oxygen that can be scavenged per moles of reactive sites can be tailored. For example, Table 2 provides a theoretical prediction of the number of moles of oxygen scavenged per molecule (as determined by the number of A rings), assuming every allyl site reacts.

TABLE 2
Theoretical Prediction - Oxygen Capacity

| | Formula of | MW of A-R₄ | moles O2/g | cc O2/g |
|---|---|---|---|---|
| No. | A-R₄ | (g/mol) | ether | ether |
| A ring starting | C₇H₁₂O | 112.2 | — | — |
| material (alcohol) | | | | |
| I | C₆H₆O | 94.1 | 0.00531 | 119.1 |
| II | C₁₂H₁₀O₂ | 186.2 | 0.00534 | 119.7 |
| III | C₁₅H₁₆O₂ | 228.3 | 0.00480 | 107.6 |
| IV | C₁₅H₁₆O | 212.3 | 0.00326 | 73.1 |
| V | C₆H₆O₂ | 110.1 | 0.00670 | 150.2 |
| VI | C₁₅H₂₄O | 220.4 | 0.00318 | 71.3 |
| VII | C₂₃H₃₂O₂ | 340.5 | 0.00378 | 84.8 |
| IX | C₅₄H₇₈O₃ | 775.2 | 0.00284 | 63.6 |

In one embodiment, the oxygen-scavenging composition is incorporated in at least one layer of a plastic article by blending the composition with any polymeric material, such as those polymeric materials commonly used in the packaging industry (e.g., polyethylene, polypropylene, PET). The polymeric material can be a passive oxygen barrier (such as EVOH), and/or a CO₂ barrier, so long as the polymeric material satisfies the structural demands required for containing oxygen-sensitive contents and optionally carbonated contents. The blend can be used in a monolayer structure or in any multilayer structure.

In one embodiment, exemplary host polymers are selected from ester-containing polymers or any suitable polyester resin having an ester in the main polymer chain. Polyesters, such as PET, are the most common materials for plastic containers. Suitable polyesters include polyethylene terephthalate (PET), polybutylene terephthalate (PBT), polypropylene terephthalate (PPT), polyethylene naphthalate (PEN), polyglycolic acid (PGA), polylactic acid (PLA), and polyhydroxyalkanoates (PHA).

Other suitable host polymers include polyacrylates (e.g., polymethyl methacrylate (PMMA), polyethylene methacrylate (PEMA), poly(methyl acrylate), poly(ethyl acrylate), poly(ethyl methacrylate)), vinyl acetates, polyolefins (e.g., polyethylene, polypropylene, polyisoprene, polybutadiene), poly(vinylalcohol), styrenic polymers (e.g., polystyrene and poly(4-methylstyrene)), polyamides (nylon-6,6, nylon 6, nylon 11, nylon-MXD6 (m-xylylene diamine) and polycaprolactam), other nitrogen-containing polymers (e.g., polyacrylamide, polyacrylonitrile and poly(styrene-co-acrylonitrile), halogenated polymers (e.g., poly(vinyl chloride), poly(vinylidene chloride) and polytetrafluoroethylene), polycarbonates (e.g., poly(4,4′-isopropylidine-diphenyl carbonate), polyethers (e.g., poly(ethylene oxide), poly(butylene glycol), poly(epichlorohydrin), and poly(vinyl butyral)), heterocyclic polymers (e.g., polyimides, polybenzimidazoles, polybenzoxazoles, and poly(vinyl pyrrolidone)), other engineering polymers (e.g., polysulfones, poly(ether ether ketones), poly(phenylene oxide), and poly(phenylene sulfide)), inorganic polymers (e.g., polysiloxanes, polysilanes, and polyphosphazenes), natural polymers and their derivatives (e.g., dextran, cellulose, and carboxymethyl cellulose), and ionomers such as sulfonate polymers (e.g., sulfonate polystyrene)), and carboxylic acid-containing polymers (e.g. copolymers of acrylic acid or methacrylic acid). Also suitable are blends or copolymers of the above including process and post-consumer regrind comprising any of the above.

In one embodiment, at least one layer of a plastic packaging material comprises the blend of oxygen scavenging composition and host polymer. Exemplary packaging materials include bottles, preforms, closures, closure liners, sheets, and films.

In one embodiment, the plastic packaging material comprises a single layer comprising the blend of oxygen-scavenging composition and host polymer, referred to herein as an “active” layer.

In one embodiment, a multilayer container comprises one or more active layers comprising the oxygen-scavenging composition incorporated in the host polymer. For example, one or two layers of polymers (such as any of the host polymers disclosed herein) that do not contain the oxygen-scavenging composition may be placed adjacent the active layer. Such a layer of polymer can be positioned to cover the active layer such that it protects the food or beverage from contacting the active layer. In one embodiment, a three layer container can be formed comprising one core layer comprising the active layer between inner and outer layers of conventional packaging polymer (e.g., PET).

In another example, a fivelayer packaging material can be formed comprising inner and outer layers of conventional packaging polymer (e.g., PET), a central core layer of a conventional packaging polymer, and first and second intermediate active layers. In a specific example, a packaging material has inner and outer layers of virgin polyester and a core layer of recycled polyester containing a compound of the present invention and a metal. In another specific example, the packaging material has inner and outer layers of virgin PET and a core layer of recycled PET containing the oxygen-scavenging composition. In yet another specific example, a packaging material has inner and outer layers of virgin polyester and a core layer of EVOH containing the oxygen-scavenging composition. Other exemplary arrangements for a wall of a packaging material, including four different materials in six layers, is described in PCT Publication No. WO 2004/106426, the disclosure of which is incorporated herein by reference. Single layer and multilayer containers can be formed by any method known in the art, such as injection molding to form a preform followed by blow molding, such as those processes described in PCT Publication No. WO 2004/106426. Other methods include compression molding and extrusion blow molding.

In one embodiment, the packaging material has at least one layer comprising the oxygen-scavenging composition and PET. This layer can be alternated with one or more layers of PET polymers or a barrier polymer, e.g., one that impedes or slows down the rate of oxygen entering a container. For example, a three layer bottle can comprise two layers of a polymer containing the oxygen-scavenging composition on alternating sides of one layer of a barrier polymer. Five layer bottles can also be constructed with three layers of polymer containing the oxygen scavenging composition, alternating with two layers of barrier polymer.

Exemplary barrier polymers include EVOH, polyamides, acrylonitrile copolymers, blends of EVOH and polyamide, nanocomposites of EVOH or polyamide and clay, blends of EVOH and an ionomer, acrylonitrile, cyclic olefin copolymers, polyvinylidene chloride (PVDC), polyethylene napthalate (PEN) polyglycolic acid (PGA), and blends thereof. In alternative embodiments, the oxygen-scavenging composition can be incorporated in a layer comprising a non-barrier polymer and positioned adjacent one or more layers of barrier polymers.

In one embodiment, the packaging materials having the oxygen scavenging compositions disclosed herein have a haze of less than 7%, such as a haze of less than 5%, or even a haze of less than 3%.

Along with containers and preforms, plastic packaging materials can include closures for containers, closure liners, sheets, and films, as described in WO 2004/106426.

In one embodiment, the layer comprising the oxygen-scavenging composition impedes or reduces the rate of oxygen ingress into the container, compared to a barrier polymer layer without the composition. In another embodiment, the oxygen-scavenging composition reduces the oxygen content inside the bottle. The oxygen content inside a bottle can be determined by any method known in the art, such as the methods described in U.S. Publication No. 2002/0037377, the disclosure of which is incorporated herein by reference.

### EXAMPLES

### Example 1

This Example describes a liquid screening test utilizing 3-cyclohexene-1-methanol for its ability to reduce the oxygen content of the atmosphere in a closed container in the presence of cobalt. 3-cyclohexene-1-methanol (5 mL) was added to a clean glass jar in neat form. The jar was then fitted with a metal closure and a rubber septum, which allowed access to the interior of the jar via a syringe needle. The seals around the metal closure and the rubber septum were then treated with an epoxy adhesive to provide a gas-tight seal. A solution of cobalt neodecanoate in n-propanol was injected into the jar to provide a concentration of the cobalt complex in the jar of 10,000 ppm.

Table 3 below shows that the allyl moiety of the cycloexene group is capable of reducing the oxygen content of the jar in comparison to 5 ml of a control liquid of n-propanol.

TABLE 3
Liquid screening test: 5 mL Liquid + 10,000 ppm CoNeo

HSO (% O₂)

| | | 3-cyclohexene-1- |
|---|---|---|
| Time (hours) | n-propanol | methanol |
| 0 | 20.8 | 20.5 |
| 6.25 | 20.9 | 10.2 |
| 20.75 | 20.8 | 8.1 |

### Example 2

This Example describes the ability of the compositions of the invention to remove oxygen from a closed container by using an oxygen headspace analysis.

Injection molded plaques were made from a blend of polymer (Phillips Sumika Marlex Polypropylene, grade HGN020-05) and the oxygen-scavenging composition. The ether was added at a concentration of 1% based on the total weight of the ether+polypropylene+cobalt neodecanoate. Cobalt neodecanoate was added at 2000 ppm or 0.2 wt % based on the total weight of the ether+polypropylene+cobalt neodecanoate, i.e., 98.8% polypropylene+1.0% additive+0.2% cobalt neodecanoate.

The plaques were placed in a jar containing water, in a platform above the water. The jar was capped with a standard canning jar lid having a rubber septum. A syringe was inserted through the septum to withdraw a gas sample from the jar; periodically, gas samples were injected into a Mocon model PacCheck 450 Head Space Analyzer to measure the oxygen content (available from Mocon Modern Controls, 7500 Boone Ave North, Minneapolis, Minn. 55428 USA). After measuring an initial oxygen content (typically 20.9%), subsequent measurements were taken over a period of several weeks.

Table 4 below shows the results of the oxygen headspace analysis. It can be seen that the compositions of the invention were effective in removing oxygen from the container (the results for compounds VI and VIII may be due to a poor seal on the jar).

TABLE 4
Oxygen Headspace Analysis

| | Time on Test | | |
|---|---|---|---|
| Ether | (days) | Initial O₂ | Final O₂ |
| I | 61 | 20.9% | 11.6% |
| II | 57 | 20.9% | 6.4% |
| III | 57 | 20.9% | 10.1% |
| IV | 57 | 20.9% | 18.2% |
| V | 57 | 20.9% | 13.8% |
| VI | 27 | 20.9% | 20.9% |
| VII | 69 | 20.9% | 11.2% |
| VIII | 69 | 20.9% | 20.9% |

These and other modifications would be readily apparent to the skilled person as included within the scope of the described invention.

1. A composition comprising:
at least one transition metal, and
a compound having the following structure: 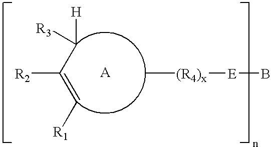
wherein:
“A” is a hydrocarbon ring,
R₁, R₂, and R₃ are independently selected from hydrogen and C₁-C₁₂ alkyl,
R₄ is an alkenyl of the formula —(CH₂)_{y}—, where y is an integer of at least 1, and x is an integer equal to 1 or 0,
E is selected from —O—, —C(═O)—O—, and —N(R)— where R is hydrogen, aryl or a branched or linear C₁-C₆ alkyl,
B is a group having a molecular weight of at least 75 g/mol, and n is an integer ranging from 1-3.

2. The composition of claim 1, wherein B is a hydrocarbon comprising at least 6 carbon atoms.

3. The composition of claim 2, wherein B is a hydrocarbon selected from alkyl, aryl, and combinations of alkyl and aryl groups.

4. The composition of claim 1, wherein R₄ is a C₁-C₁₂ alkenyl.

5. The composition of claim 1, wherein A is a C₆ ring.

6. The composition of claim 1, wherein n=1.

7. The composition of claim 1, wherein n=2 or 3.

8. A plastic packaging article having at least one layer comprising a polymeric material blended with an the oxygen-scavenging composition, the composition comprising:
at least one transition metal, and
a compound having the following structure: 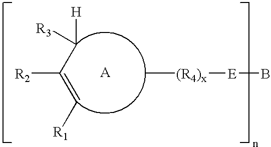
wherein:
“A” is a hydrocarbon ring,
R₁, R₂, and R₃ are independently selected from hydrogen and C₁-C₁₂ alkyl,
R₄ is an alkenyl of the formula —(CH₂)_{y}—, where y is an integer of at least 1, and x is an integer equal to 1 or 0,
E is selected from —O—, —C(═O)—O—, and —N(R)— where R is hydrogen, aryl or a branched or linear C₁-C₆ alkyl,
B is a group having a molecular weight of at least 75 g/mol, and n is an integer ranging from 1-3.

9. The article of claim 8, wherein the polymeric material comprises a polymer selected from polyesters, polyolefins, polyacrylates, polyvinyl acetates, styrenic polymers, EVOH, polyamides, polyacrylamides, polyacrylonitriles, poly(styrene-co-acrylonitrile), poly(vinyl chloride), poly(vinylidene chloride), polytetrafluoroethylene, polycarbonates, polyethers, polyimides, polybenzimidazoles, polybenzoxazoles, poly(vinyl pyrrolidone)), polysulfones, poly(ether ether ketones), poly(phenylene oxide), poly(phenylene sulfide), polysiloxanes, polysilanes, polyphosphazenes), dextran, cellulose, carboxymethyl cellulose, and sulfonate polymers

10. The article of claim 9, wherein the polyesters are selected from PET.

11. The article of claim 8, wherein the article is a monolayer article.

12. The article of claim 8, wherein the article is a multilayer article.

13. The article of claim 8, wherein the article further comprises at least one polymer layer free of the oxygen-scavenging composition positioned adjacent the at least one layer containing the oxygen-scavenging composition.

14. The article of claim 13, wherein the polymer layer free of the oxygen-scavenging composition are positioned adjacent either side of the at least one polymer layer containing the oxygen-scavenging composition.

15. The article of claim 14, wherein the at least one layer of containing the oxygen-scavenging composition comprises PET.

16. The article of claim 14, wherein one or more layers, including the at least one layer free of the oxygen-scavenging composition, comprises a barrier polymer.

17. The article of claim 16, wherein the barrier polymer is selected from EVOH, polyamides, acrylonitrile copolymers, blends of EVOH and polyamide, nanocomposites of EVOH or polyamide and clay, blends of EVOH and an ionomer, acrylonitrile, cyclic olefin copolymers, polyvinylidene chloride (PVDC), polyethylene napthalate (PEN) polyglycolic acid (PGA), and blends thereof.

18. The article of claim 14, wherein the at least one layer of free of the oxygen-scavenging composition comprises PET.

19. The article of claim 14, wherein the article is a monolayer article.

20. The article of claim 14, wherein the article is a multilayer article.

21. The article of claim 1, wherein the article is selected from preforms, containers, closures, closure liners, films, and sheets.

22. An oxygen-scavenging composition, comprising:
a nonpolymeric compound comprising a ring, three of the ring carbon atoms forming an allyl group with an allylic hydrogen, the ring being bonded via a ring substituent to an ether or carboxylic linkage, the ether or carboxylic linkage being further bonded to a group having a molecular weight of at least 75 g/mol; and
at least one transition metal.

23. A plastic packaging article having at least one layer comprising the oxygen-scavenging composition of claim 22, wherein the article is selected from preforms, containers, closures, closure liners, films, and sheets.

24. A compound having the following structure: 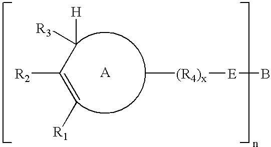
wherein:
“A” is a hydrocarbon ring,
R₁, R₂, and R₃ are independently selected from hydrogen and C₁-C₁₂ alkyl,
R₄ is an alkenyl of the formula —(CH₂)_{y}—, where y is an integer of at least 1, and x is an integer equal to 1 or 0,
E is selected from —O—, —C(═O)—O—, and —N(R)— where R is hydrogen, aryl or a branched or linear C₁-C₆ alkyl,
B is a group having a molecular weight of at least 75 g/mol, and n is an integer ranging from 1-3.
